(19) United States
(12) Patent Application Publication
(10) Pub. No.: US 2008/0275396 A1
(43) Pub. Date: Nov. 6, 2008

(54) Cannula Inserting System

(75) Inventors: Sieglinde Neerken, Eindhoven (NL); Gerhardus Wilhelmus Lucassen, Eindhoven (NL); Hendricus Johannes Maria Sloot, Eindhoven (NL); Carole Schwach, Eindhoven (NL); Frederikus Johannes Maria De Vreede, Eindhoven (NL); Robertus Hekkenberg, Oude Wetering (NL); Marion Geerligs, Eindhoven (NL); Nicole Leonarda Wilhelmina Eikelenberg, Eindhoven (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/913,696

(22) PCT Filed: May 4, 2006
(86) PCT No.: PCT/IB2006/051408

§ 371 Date: Nov. 6, 2007

(30) Foreign Application Priority Data
May 10, 2005 (EP) 05300361.2

(51) Int. Cl. A61M 5/42

(52) U.S. Cl. 604/116

(57) ABSTRACT

The present invention provides a highly automated puncture system for inserting a cannula or a needle into a blood vessel of a person or an animal. The puncture system has an acquisition module that allows for determining at least a location of a blood vessel underneath of the surface of a skin and is further enabled to determine an optimal puncture location that is well suitable for inserting a cannula into the blood vessel. Further, the puncture system has an actuator for moving and aligning the cannula to a determined position. The system is further adapted to autonomously insert the cannula into the blood vessel for multiple purposes, such as blood withdrawal, venous medication and infusions.

The present invention relates to the field of cannulation, hence to the insertion of a cannula or needle into the vascular system of a person or an animal.

Insertion of a cannula or a needle into a person's vascular system is an everyday task for physicians and has to be performed with high accuracy and care. Therefore, medical personnel has to be highly skilled for such tasks as blood withdrawal, drug delivery or infusions. The physician has to find an appropriate blood vessel and to introduce a distal end of a cannula or a needle with a very high precision in order to prevent generation of hematoma or effusions. Depending on the vascular system of a person even a highly skilled and experienced physician may require several attempts to insert a needle or a cannula at a suitable location into a blood vessel. Such multiple attempts of puncturing are rather painful and cause appreciable patient discomfort. Moreover, such multiple attempts are also rather time intensive, which is disadvantageous especially in emergency situations.

There exist various devices and systems providing needle or cannula guiding and that assist a physician for inserting the cannula or needle in the vascular system of a patient. The document US 2003/0060716 discloses a device for inserting the distal end of a hollow needle into a blood vessel. There is provided location means as an aid for locating a vessel and a holder for the cannula to be introduced. Also the introduction procedure is mechanized in that for an indwelling cannula and for a puncture cannula in each case there is provided a separate holder, which are traversable along a guide independently of one another in the direction of introduction as well as in the counter direction. In order to provide the physician with an aiding position on aligning the device to the patient, there is provided an optical or acoustic display which emits a signal when the device is located in the designated position for puncture of the located vessel. The device is moved manually over the skin until the display lights up, thereby indicating that an optimal position of the device to the vessel is reached for introducing the cannula.

Even though the disclosed device provides mechanized introduction of a cannula into a blood vessel, alignment and positioning of the device to the patient and to the located blood vessel has to be performed manually. Furthermore, during introduction of a cannula the device must not be moved or tilted, which requires respective skills of the physician or medical personnel. Also, the device only indicates, that an optimal position for introducing the cannula has been reached, but does not provide any active guidance for finding this optimal position. Hence, the physician making use of the device requires at least profound skills and experience for finding the optimal position of the device for introducing of the cannula.

Many applications need to differentiate whether the blood vessel to be punctured is an artery or a vein. U.S. Pat. No. 6,755,789 B for example discloses an apparatus for the cannulation of blood vessels. The apparatus uses a transducer array to obtain an ultrasound image of tissue, and Doppler transducer elements to determine whether the blood vessel to be punctured is an artery or a vein. Puncturing of the blood vessel is done manually by medical staff.

It is an object of the present invention to provide a highly automated needle and cannula inserting usable both for semi-automatic and automatic use which can be used in an increased range of applications with increased safety.

The present invention provides a puncture system for inserting a needle or cannula into a blood vessel of a person or an animal. The puncture system provides highly automated cannulation, i.e. insertion of a cannula or needle into a vascular system of a patient and comprises location determination means providing at least a location of the blood vessel. The system further comprises processing means for determining a puncture location of the blood vessel, hence an optimal location of the blood vessel that is suitable for a needle or cannula insertion. This puncture location is determined by making use of output signals of the location determination means. The puncture system further has cannula insertion means for fixing the needle or cannula with respect to the puncture system. The cannula insertion means are moveable with respect to an inserting direction and at least with respect to a second direction that is substantially non-parallel to the inserting direction.

The inserting direction is typically given by the alignment of the needle or cannula and determines an angle at which the needle or cannula is intended to emerge the vascular system. Further, the cannula insertion means are also moveable along at least a second direction, such as e.g. a direction being substantially parallel to the surface of the skin of the person or animal. Hence, the needle or cannula is moveable with respect to the location determination means as well as with respect to a blood vessel. It is however possible as well that the localisation determination means have a spatially fixed relationship to the cannula. In this case the localization determination means is rigidly connected to the cannula such that they represent a rigid system which is be moveable with respect to the blood vessel.

Furthermore, the cannula insertion means provides manual movement and alignment of the needle or cannula and provide manual insertion of the distal end of the needle or cannula into the blood vessel at the puncture location. In essence, this provides semi automated insertion of the needle or cannula. The puncture system autonomously allocates and determines a suitable puncture location and guides an operator to insert the needle or cannula at the specified location into the blood vessel. The actual insertion is performed operator supported. The force required for inserting the needle or cannula into the blood vessel is then provided by the operator, thus guaranteeing a maximum of sensitivity during needle or cannula insertion.

For instance, the location determination means and the processing means serve to locate and to identify a blood vessel and to determine the puncture location, but insertion of the needle or cannula is not performed in a completely automated but in a way that is at least partially controlled by the operator. For example, the puncture system may direct the needle or cannula along the inserting direction and may also translate the cannula to the inserting position whereas insertion of the cannula or needle, i.e. translation of the cannula or needle along the inserting direction is performed operator supported.

Typically, the location determination means is adapted to provide a plurality of geometric data of the blood vessel, which allows to determine parameters, such like blood vessel diameter, blood vessel size as well as a depth under the skin. Further, the location determination means effectively provide determination of the blood vessel's course. Making effective use of such geometric and location information of the blood vessel allows to determine an optimal puncture location with a high accuracy and reliability that finally allows to minimize a danger of injury of a vessel wall. Consequently generation or severity of bleeding, hematoma or inflammation can be reduced to a minimum. Also by effective usage of obtained geometric and location data of the blood vessel, multiple attempts for a needle or cannula insertion can be prevented, because the reliable and accurate inspection of the blood vessel prior to insertion of the needle or cannula nearly guarantees that the needle or cannula can be correctly inserted or introduced into the vascular system with a single attempt. In particular, in emergency situations it is obvious, that this guided puncture is highly advantageous compared to an entirely manual cannulation.

The blood vessel identification means are adapted to identify whether a blood vessel is an artery or a vein. With this additional functionality the cannula insertion system is safer to use as many applications require a puncturing of the correct blood vessel type. This makes it possible that less experienced medical staff use the cannula inserting system, an aspect which saves money within the expensive health system. It is even conceivable that patients having no medical knowledge use the cannula insertion system under the supervision of medical staff. Blood withdrawal would then become as easy as the act of urination.

A first possibility for identifying the blood vessel type consists in applying conventional Doppler techniques with or without imaging, in particular with an ultrasonic or optical Doppler system. In this case an ultrasound or optical signal is coupled into the tissue containing the blood vessel and then absorbed by particles in the blood. The ultrasound or optical energy is then emitted by the particles and detected by a sensor as a Doppler signal. Blood flowing away from the sensor emits ultrasound or optical waves having a frequency being lower than the waves being coupled into the tissue. The Doppler signal thus yields the direction of the blood flow which distinguishes arteries from veins, as blood in arteries is flowing away from the heart, and blood in veins is flowing towards the heart. This flow direction of a blood vessel can be marked by a colour. The direction of the blood flow might be assigned the colour red or blue, indicating a flow towards or away from the ultrasound transducer or optical probe. This is why this technique is called Colour Doppler (Ultrasound) technique.

A second possibility similar to the first one is to determine the frequency shift of the Doppler signal as a function of time. The result can be used to calculate the blood flow as a function of time. The flow in a vein is rather constant in time, whereas the flow in an artery is pulsating in nature, the frequency of the pulses representing the heart rate. Thus the pulsating or non-pulsating nature of the blood flow can be used to distinguish an artery from a vein.

A third possibility is to carry out a mechanical palpation. If the tissue containing the blood vessel is subjected to a mechanical pressure veins have the tendency to collapse, whereas arteries do not collapse due to different vessel wall characteristics. Thus the different behaviour of veins and arteries with respect to mechanical pressure can be used to distinguish arteries from veins. The mechanical palpation can be carried out by pushing an imaging probe onto the skin and to follow the corresponding signal.

A fourth possibility do distinguish arteries from veins is the oxygen content in the blood which can be measured by an absorption technique. In a first step the blood vessel is subjected to light of a first wavelength which is well absorbed by low-oxygen blood found in veins. In a second step the blood vessel is subjected to light of a second wavelength which is well absorbed by high-oxygen blood found in arteries. Thus measuring and analyzing the absorption of the two wavelengths allows to distinguish arteries from veins.

According to a further embodiment, the location determination means are not only adapted to detect and to identify a blood vessel underneath the skin surface but also provide location determination of the cannula's distal end. Hence, by means of the location determination means, it can be precisely checked whether the cannula or needle has been correctly inserted into the vascular system of the person or animal.

Additionally, the puncture system features respective indication means that are adapted to indicate whether the cannula or needle has been correctly inserted into the blood vessel by the puncture system.

According to a further embodiment of the invention, the location means are further adapted to track the location of the needle's or cannula's distal end during insertion of the needle or cannula. The puncture system further has control means for controlling the movement of the needle or cannula in response to the tracking of the needle's or cannula's distal end. In this way, the puncture system is provided with a feedback allowing to monitor and to check whether the distal end of the needle or cannula is correctly inserted. This functionality effectively represents a safety mechanism of the puncture system and helps to prevent that despite of an accurate inspection of the blood vessel the cannula might be incorrectly introduced, which may have serious consequences for the person's or animal's health.

Typically, the location determination means provide course and location determination of the blood vessel as well as tracking of the needle's or cannula's distal end at a sufficient repetition rate that allows for fast reaction in case that the cannula introduction deviates from a determined path or schedule. Also, the location determination means allow to check whether the distal end of the needle or cannula has been inserted correctly into the persons vascular system. Hence, the location determination means not only provide a control mechanism during needle or cannula insertion but also allow to check the final position of the needle or cannula after the intra vascular inserting has been terminated.

Instead of tracking the needle's or cannula's distal end it is also possible to monitor and follow the position or movement of the blood vessel during insertion. In principle this should also provide enough information and is a somewhat simpler solution. If it is known where the needle or cannula has to end up and if the insertion parameters have been determined, the location of the blood vessel can be monitored during insertion. The insertion however goes wrong if the blood vessel does not stay in place.

According to a preferred embodiment, the puncture system further comprises actuation means that are adapted to autonomously move the cannula insertion means into an inserting position and that are further adapted to insert the distal end of the needle or cannula into the blood vessel at the puncture location. In this embodiment the invention provides an entirely automated needle or cannula insertion. Hence, the inventive puncture system provides a location determination of the blood vessel, an autonomous alignment and movement of the cannula to an inserting position and inserting direction and finally an automated inserting of the cannula into the blood vessel at a location determined by the puncture system itself.

In this sense the inventive puncture system provides an entirely automated insertion of a needle or cannula into a person's or animal's blood vessel, which is applicable to e.g. blood sampling or blood withdrawal, drug medication or infusion, blood transfusion, general catheter insertion and dialysis. Preferably, the entire process of locating of a blood vessel, determining of a puncture location as well as mechanically shifting and aligning the needle or cannula and finally inserting of the needle or cannula can be performed without any user interaction, allowing to execute the above mentioned tasks in a highly automated manner that may even be performed by unskilled or low-skilled medical personnel.

According to a further embodiment, the actuation means are manually controllable by an operator for manual insertion of the cannula by the operator. Even if implemented as an autonomous system, the puncture system also allows for a partially automated insertion of a needle or cannula.

In a further preferred embodiment, the invention comprises fixing means for attaching the puncture system to the surface of the skin of the person or of the animal. Typically, these fixing means are designed as a cuff or collar that can be rigidly attached to e.g. an arm or arm bend of a person but also to other body parts of a person or animal that are suitable for e.g. blood withdrawal or infusions like the back of the hand. However, application of the needle insertion system is by no means restricted to the above mentioned parts of a body. In particular, by making effective use of the automated and high precision blood vessel location determination, other application scenarios become conceivable. For instance, the needle or cannula inserting system may become applicable to body sites that are not yet accessible by manual puncturing performed by educated medical personnel. Since the fixing means provide rigid attachment of the puncture system to a suitable body part, the cannula insertion means are moveable with respect to these fixing means in order to bring the needle or cannula into the determined inserting position.

According to a further preferred embodiment of the invention, the location means comprise optic and/or acoustic or opto-acoustic detection or signaling means and corresponding processing means that are adapted to perform a corresponding signal acquisition and signal processing for deriving relevant parameters for determination of the puncture location. The detection or signaling means may be realized as imaging and image processing means but may also be implemented as an acquisition system making use of e.g. Doppler based techniques that do not require image acquisition and respective image processing.

Typically, the location determination means are realized by making use of techniques, such as Near infrared imaging, Optical Coherence Tomography, Photo Acoustic Imaging or Ultrasound Techniques. In particular, Ultrasound techniques, Optical Coherence Tomography and Photo Acoustic Imaging provide inspection and analysis of the blood vessel and allow for a precise acquisition of blood vessel related data, even if the blood vessel is located at an appreciable depth under the skin of the person. Other techniques, that might be based on Doppler signals, like Doppler Ultrasound or Doppler Optical Coherence Tomography that are adapted to locate a flowing or streaming liquid, such as blood flowing in a blood vessel, in principle also allow a precise and reliable location determination of a blood vessel underneath the skin surface. Also, combinations of Doppler-based and imaging based signal acquisition techniques might be implemented.

In a further embodiment, the processing means are further adapted to determine the inserting position in response to the puncture position, which in turn is obtained by means of an optimization procedure performed by the processing means that accounts for at least one blood vessel parameter that is derived from a data acquisition performed by the location determination means. The optimization procedure is preferably performed by making use of a plurality of blood vessel parameters, such as blood vessel size, blood vessel diameter, location and depth underneath the skin surface, the course of the blood vessel as well as the general geometry of the blood vessel. For example, the processing means are supplied with graphical data processing means in order to recognize and identify particular regions of a blood vessel that are not suitable for puncture, such as regions of a blood vessel that are near a branch or a junction. Also, the processing means and the location determination means may determine regions of a blood vessel featuring a narrowing that might be due to calcification. Hence, the optimization procedure serves to determine an optimal location of the blood vessel that is suitable for puncturing and that is expected to cause a minimum of discomfort or pain.

The optimization procedure determines the optimal puncture position of the blood vessel but it may further be applied to determine a corresponding inserting position for the cannula that is governed by the puncture position as well as by the inserting direction of the cannula. The inserting direction, hence the angle at which the needle or cannula is to be inserted into the blood vessel, is also preferably determined by means of the optimization procedure and typically varies between 10° and 45° with respect to the person's skin. Depending on the blood vessel's depth underneath the skin surface and the inserting direction, the inserting position may substantially deviate from the puncture position of the blood vessel.

According to a further preferred embodiment, the orientation of the needle or cannula and the inserting direction are modifiable and the actuation means are adapted to translate and to align the cannula insertion means in three spatial directions. In this way, the needle or cannula may be freely arranged and oriented within a range of coverage of the puncture system and may therefore be arbitrarily positioned as determined by the processing means and the puncture location.

According to a further preferred embodiment, the needle or cannula is applicable to blood withdrawal and/or drug infusion and/or blood transfusion, and/or catheter insertion and/or dialysis applications. Hence, the invention can be universally applied to various different medical purposes that require insertion of a needle or cannula into a vascular system of a person. Respective cannula insertion means for fixing the needle or cannula are typically realized by making use of a modular concept allowing for a quick and secure adaptation of the needle or cannula inserting system to a multitude of different purposes.

In another preferred embodiment, the invention provides autonomous insertion of the needle's or cannula's distal end into the blood vessel in response of detecting that the needle or cannula is at the insertion position. Hence, insertion of the needle or cannula is typically performed by means of a two step process. In a first step the needle or cannula is translated and moved as well as aligned in the inserting position and along the inserting direction and thereafter in the second step any movement of the needle or cannula is disabled except for a movement along the inserting direction. In this way, it can be effectively guaranteed, that during cannula insertion a potentially harmful lateral movement of the needle or cannula is prevented. Hence, during cannula insertion the lateral position of the cannula remains substantially fixed with respect to the surface of the skin, i.e. the needle or cannula is only moved along the inserting direction.

In another aspect, the invention provides a computer program product for a puncture system that is adapted to insert a needle or cannula into a blood vessel of a person or animal. The computer program product is executable by processing means of the puncture system and is further operable to perform a determination of at least one blood vessel parameter of the blood vessel by processing of the output of location determination means. The computer program product is inherently adapted to recognize and to identify a blood vessel. It is further enabled to exploit the blood vessel recognition and identification for acquiring the at least one relevant blood vessel parameters. This at least one blood vessel parameter is at least representative of a blood vessel's location underneath the surface of the skin. Preferably, a plurality of blood vessel parameters representing blood vessel size, blood vessel diameter as well as a course of the blood vessel, geometry of the blood vessel and depth underneath the surface of the skin can be precisely determined. The computer program product is further operable to determine a puncture location of the blood vessel by processing of the at least one blood vessel parameter. Preferably, by making use of a plurality of blood vessel parameters, the computer program product is operable to perform an optimization procedure in order to determine an optimal puncture location of the blood vessel.

Further, the computer program product is operable to calculate a driving signal for driving actuation means of the puncture system in order to insert the needle's or cannula's distal end into the blood vessel at the puncture location. In this way, the computer program product allows to analyze acquired blood vessel data in order to determine an optimal puncture location and to control mechanical drives and actuators for inserting the needle's or cannula's distal end into the blood vessel in a precise, reliable and less painful way compared to manual needle or cannula insertion performed by medical personnel or by a physician.

In still another aspect the invention provides a puncture system that comprises a cuff for fixing the puncture system to a body part of a person or animal and an apparatus for detecting a blood vessel underneath the surface of the skin of the body part and for autonomously inserting a needle or cannula into the blood vessel in response to detect at least the blood vessel's location. The autonomous insertion of the cannula comprises acquisition of geometric data of the blood vessel, determination of a puncture location of the blood vessel and a corresponding positioning and aligning of the needle or cannula for inserting the distal end of the cannula into the blood vessel at the puncture location. Therefore, the puncture system comprises an acquisition system for acquiring location, course and also other geometric data of the blood vessel. Further, the system has a processing unit for determining a puncture location of the blood vessel and at least an actuator for moving and aligning the needle or cannula in an appropriate way.

In a further preferred embodiment, the system further comprises a movement control unit that is adapted to process data provided by the acquisition system during insertion of the cannula in order to control the needle's or cannula's movement during insertion into the tissue of the person or animal.

In a further embodiment, the puncture system is adapted to insert the needle or cannula and to be detached from the inserted needle or cannula in such a way, that the puncture system is removable from the body part leaving the needle or cannula inserted in the biological tissue, e.g. in the skin. Therefore, the cannula insertion means are adapted to dissociate the needle or cannula, if the needle or cannula has been correctly inserted. Further, if the needle or cannula is correctly inserted, also the cuff can be loosened thus allowing to detach the entire puncture system from the body part. This feature is particularly advantageous with respect to catheter as well as infusion related application scenarios.

Further, it is to be noted that any reference signs in the claims are not to be construed as limiting the scope of the present invention.

### BRIEF DESCRIPTION OF THE DRAWINGS

In the following various embodiments of the invention will be described in greater detail by making reference to the drawings in which:

FIG. 1 illustrates a schematic block diagram of the inventive puncture system,

FIG. 2 shows the blood vessel identification by means of the Colour Doppler technique,

FIG. 3 shows the blood vessel identification by means of the Pulsed Doppler technique,

FIG. 4a,

FIG. 4b show the blood vessel identification by means of mechanical palpation,

FIG. 5 shows absorption spectra of Hb and HbO₂,

FIG. 6 shows a schematic illustration of puncture location and inserting position determined by the puncture system,

FIG. 7 shows attachment of the puncture system to a suitable body part,

FIG. 8 shows a flowchart of determining puncture location and performing needle or cannula insertion,

FIG. 9 shows a flowchart of needle or cannula insertion with skin penetration detection.

### DETAILED DESCRIPTION OF THE DRAWINGS AND OF THE PREFERRED EMBODIMENTS

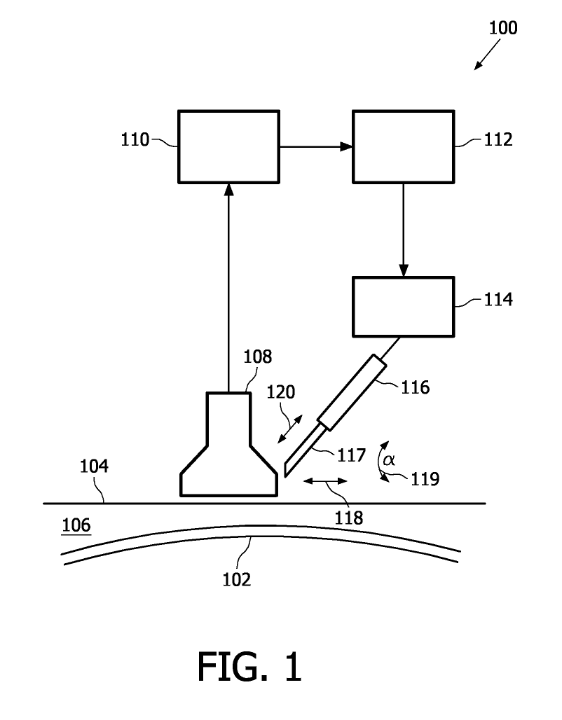

FIG. 1 shows a schematic block diagram of the puncture system 100. The puncture system 100 has an acquisition module 108, a detection system 110, a control unit 112, a cannula control 114 as well as a cannula mount 116. The cannula 117 itself can be rigidly attached to the cannula mount 116 that represents cannula insertion means for fixing the cannula and means for moving and aligning the cannula 117 as controlled by the cannula control unit 114. The cannula 117 and the cannula mount 116 can be moved along the inserting direction 120 as well as along direction 118 that is substantially parallel to the surface of the skin 104. In principle direction 118 can be any direction in the plane parallel to the skin surface. Typically, the cannula 117 and the cannula mount 116 are moveable by means of the cannula control 114 in all three spatial directions. Also, the angle α 119 between the inserting direction 120 and the surface of the skin 104 may be arbitrarily modified by means of the cannula control 114 in a way that is determined by means of the detection system 110 and the control unit 112.

FIG. 1 shows application of the puncture system to a person by means of a cross sectional illustration of the person's skin 104. Underneath the surface of the skin 104 there is located a blood vessel 102 that is surrounded by tissue 106. When the puncture system 100 is attached to the skin 104 of the person, the acquisition module 108 is adapted to acquire optical, opto-acoustic or acoustic data from the tissue 106 and the blood vessel 102 that allows to classify at least one blood vessel parameter, such as location of the blood vessel, diameter of the blood vessel, size of the blood vessel, depth underneath the surface of the skin 104, geometry of the blood vessel, blood flow or similar parameters.

Preferably, the acquisition module 108 is realized by means of Ultrasound, Near-infrared imaging, Optical Coherence Tomography, Doppler Ultrasound, Doppler Optical Coherence Tomography or Photo Acoustic techniques that allow to generate a signal providing identification of the blood vessel 102. Signals acquired by the acquisition module 108 are provided to the detection system 110, which in turn generates a signal of the blood vessel 102. Hence, detection system 110 as well as acquisition module 108 are coordinated in a sense that the detection system 110 is suitable to perform signal processing of signals obtained from the acquisition module 108. By making use of optical, opto-acoustic or ultrasound detection, the blood vessel 102 may be precisely located even at an appreciable depth underneath the surface of the skin 104. Additionally or alternatively also Doppler techniques may be applied including e.g. Doppler Ultrasound techniques allowing for detection of e.g. blood flow in the blood vessel 102. Also, Doppler Optical Coherence Tomography might be correspondingly applied.

Acquisition of location data, geometric data as well as data related to the course of the blood vessel 102, may also be obtained without an imaging of the blood vessel. Therefore, the imaging system 110 does not necessarily have to provide a visual image. Instead the imaging system 110 may be enabled to directly extract blood vessel parameters from the signals acquired by the acquisition module 108. Hence, extraction of blood vessel parameters may be performed by means of the detection system 110 or by the control unit 112

The control unit 112 has a processing unit that is enabled to process the data obtained from the detection system 110. Depending on the type of data provided by the detection system 110, the processing unit of the control unit 112 may further process blood vessel parameters in order to extract required blood vessel parameters from a signal of the blood vessel 102.

The control unit 112 serves to process the blood vessel parameters in order to find and to determine a puncture location of the blood vessel 102 that is ideally suited for an insertion of the cannula 117. In a basic embodiment this puncture location may be determined with respect to location and course of the blood vessel 102. More sophisticated implementations further account for the vessel geometry in the vicinity of an intended puncture location as well as vessel diameter and depth underneath the surface of the skin 104.

Typically, the puncture location may be determined as a result of an optimization procedure taking into account all kinds of blood vessel parameters. For instance, the optimization procedure that is typically performed by means of the processing unit of the control unit 112 may specify, that a puncture location must not be in the vicinity of a branch or junction of a blood vessel 102. Further, a puncture location may require a certain diameter of the blood vessel 102. Also, the puncture location may be determined with respect of a smallest possible depth of the blood vessel 102 underneath the surface of the skin 104. Additionally, the control unit may also determine the inserting direction 120 specifying at which angle α 119 the cannula 117 has to be introduced into the skin 104 and the tissue 106.

Having determined the puncture location, the control unit 112 is further adapted to specify an inserting position for the cannula 117. The inserting position specifies a position as well as an alignment or direction of the cannula 117 from which the cannula 117 has to be shifted along the inserting direction, i.e. the direction coinciding with the longitudinal direction of the cannula, in order to impinge into the blood vessel at the determined puncture location with its distal end.

After determination of puncture location and inserting position, the control unit 112 generates corresponding driving signals to the cannula control 114, which in turn is adapted to move the cannula mount 116 into a corresponding position. The cannula control 114 is typically rigidly connected to the cannula mount 116 and is provided with e.g. electro-mechanical means, such as electromotive actuators, like e.g. piezo-driven actuators that allow for a precise electronically controlled mechanical movement of the cannula 117. Typically, the cannula control 114 provides lateral displacement of the cannula mount 116 as well as shifting the cannula mount 116 or the cannula 117 along the inserting direction 120.

As soon as the cannula 117 advances towards the blood vessel 102, the acquisition module 108 also acquires position data of the distal end of the cannula 117. Especially, when the cannula 117 already penetrated the skin 104, detection of its distal end allows to control the movement of the cannula 117 through the tissue. As soon as the acquisition module 108 detects, that the distal end of the cannula 117 does not properly hit the blood vessel 102, the entire process of cannula insertion may be aborted and the cannula 117 might be withdrawn. In this way, simultaneous acquisition of blood vessel related data and position data of the distal end of the cannula 117 allows to effectively realize a feedback and security mechanism for the autonomous puncture system.

Instead of tracking the needle's or cannula's distal end 122 it is also possible to monitor and follow the position or movement of the blood vessel 102 during insertion which is a simpler solution than the one described in the last paragraph. If it is known where the needle or cannula 117 has to end up and if the insertion parameters have been determined, the location of the blood vessel 102 can be monitored during insertion. The insertion however goes wrong if the blood vessel moves.

Since the puncture system 100 is principally applicable to any kind of puncture related applications, like blood sampling, blood withdrawal, infusion or medication, the cannula can be effectively replaced by a needle or catheter that are specific for dedicated applications, respectively.

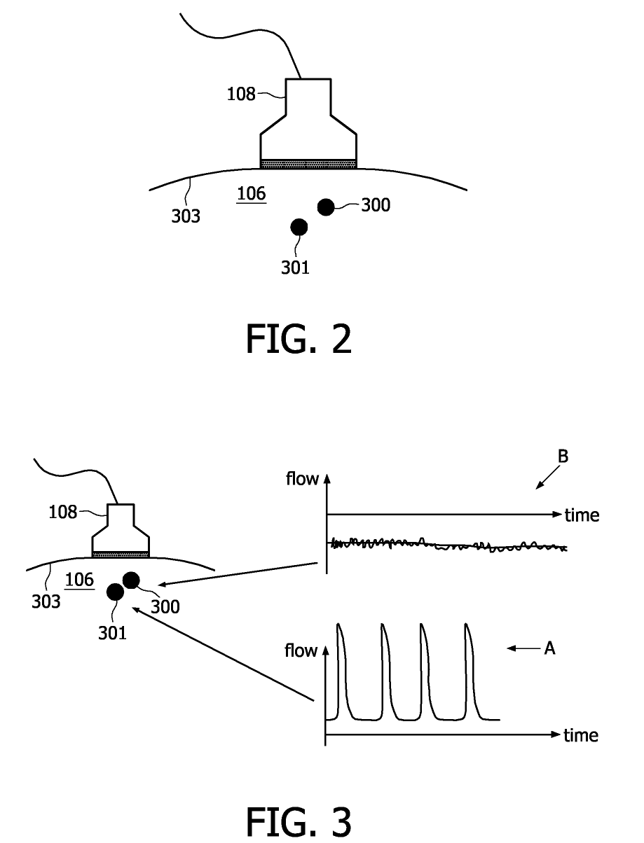

FIG. 2 shows the blood vessel identification by means of the Colour Doppler technique. In this case the blood vessel identification means is identical to the acquisition module 108, i.e. the acquisition module 108 is adapted to distinguish whether the blood vessel is an artery 301 or a vein 300. In this way the flow direction in the blood vessel identification is indicated by a colour. As an example, the colour red might be used for blood flowing away from the heart, hence for an artery 301. The colour blue is used for blood flowing towards the heart, hence for a vein 300.

FIG. 3 shows the blood vessel identification by means of the Pulsed Doppler technique. Again the blood vessel identification means is identical to the acquisition module 108, i.e. the acquisition module 108 is adapted to distinguish whether the blood vessel is an artery 301 or a vein 300. In the case of blood flowing in an artery 301, a plot of the flow rate versus the time (case A) yields pronounced peaks, whereby the reciprocal time difference between two adjacent peaks represents the heart rate. In the case of a vein 300 the corresponding plot (case B) shows a rather constant flow rate.

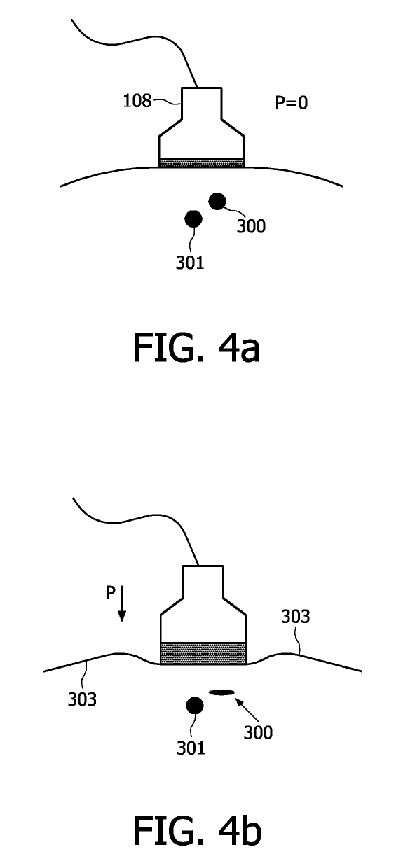

FIGS. 4a and 4b shows the blood vessel identification by means of mechanical palpation. In FIG. 4a the acquisition module 108 detects an artery 301 or a vein 300. In the case of FIG. 4a these two blood vessels have a direction roughly perpendicular to the plane of the sheet of paper on which the figure can be found (the same applies to FIGS. 2, 3, and 4b). The acquisition is carried out without exerting a pressure P on the skin 106 which contains the blood vessels 300, 301. Hence P=0. In the case of FIG. 4b the acquisition module 108 is pressed against the skin as indicated by the arrow and the lowering of the acquisition module 108 in comparison to the skin surface 303 on the left hand side and on the right hand side of acquisition module 108. The pressure has no effect on the geometry of artery 301. However, vein 300 collapses, i.e. decreases its cross section due to the pressure P. This collapsing or non-collapsing can be detected by appropriate means, for example by an Ultrasound or optical technique. In this case the flow rate of an artery 301 remains roughly constant under pressure, whereas it decreases significantly in the case of a vein 300.

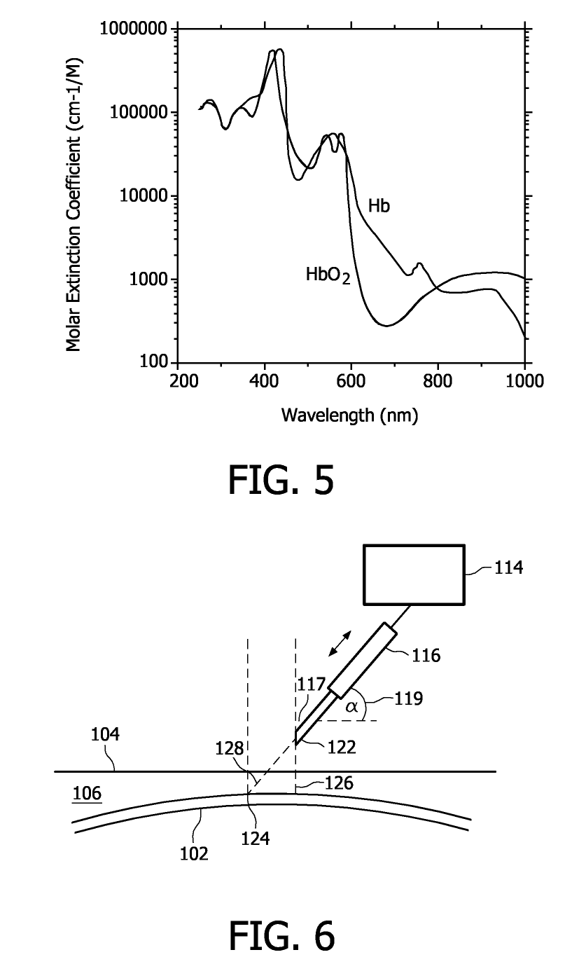

FIG. 5 shows the absorption spectra of deoxy-hemoglobin (Hb) and oxy-hemoglobin (HbO₂). In this figure the molar extinction coefficient (in units of cm⁻¹ mol⁻¹ 1=cm⁻¹ M⁻¹) is plotted versus the wavelength (in nanometers) of light. In the wavelength range of 600 nm to 800 nm the absorption differences of oxygenated blood (of an artery 301) and de-oxygenated blood (of a vein 300) can be used to distinguish between arteries and veins.

FIG. 6 shows a schematic illustration of puncture location 124 and the inserting position 126 that are determined by the puncture system. Similar to FIG. 1 also a cross sectional view of a person's or animal's skin is shown. The puncture system 100 determines the puncture location 124 of the blood vessel 102 by making use of blood vessel parameters that were obtained by means of the acquisition module 108 and corresponding detection and processing means. Here, the blood vessel 102 shows a uniform diameter and the puncture location 124 is determined by a position of the blood vessel 102 that is closest to the surface of the skin 104. This puncture location 124 may also be chosen by an experienced physician for manually inserting the cannula into the blood vessel 102. Hence, the inventive puncture system aims to determine a puncture location that provides a minimum of discomfort and pain as well as a minimum of danger of injury to the vessel wall, which may have severe consequences for the health status of the person.

Furthermore, the control unit 112 may also determine an optimal insertion angle α 119 that determines the insertion direction 120 of the cannula 117. Since the cannula 117 is typically introduced at a non-perpendicular angle with respect to the surface of the skin 104, the point of penetration through the skin 104 and the puncture location 124 typically describe an insertion path 128 for the cannula 117 that coincides with the inserting direction 120. Before advancing the cannula 117 into the skin 104, it has to be moved to the inserting position 126 featuring a lateral displacement from the puncture location 124. In this context, lateral displacement is to be interpreted as a displacement in the plane of the surface of the skin 104. For instance, the inserting position 126 may be determined as the position where the distal end of the cannula 122 coincides with the insertion path 128.

Insertion of the needle is preferably performed as a two step process, wherein the first step is given by moving the cannula 117 to the inserting position 126 by means of the cannula control unit 114. As soon as the inserting position 126 has been reached by the distal end of the cannula 122, the second step of advancing and inserting the cannula 117 into the skin 104, the tissue 106 and finally into the puncture location of the blood vessel 102 is initialized. Advancing and inserting of the cannula 117 is controlled by means of the acquisition module 108 and the control unit 112 in order to correct the movement of the cannula 117 during the insertion process. However, as soon as the acquisition module 108 detects, that the distal end of the cannula 122 has penetrated through the surface of the skin 104, a lateral movement of the cannula by means of the cannula control 114 is disabled for preventing severe injury of the skin 104 and the tissue 106.

Additionally, the puncture system 100 may be provided with a release module in order to manually control the insertion of the cannula 117. Then, a user of the puncture system may manually trigger the advancing of the cannula 117 along the inserting direction 120 and may manually control whether the inserting position 126 and/or the puncture location 124 determined by a puncture system are reasonable for inserting a cannula.

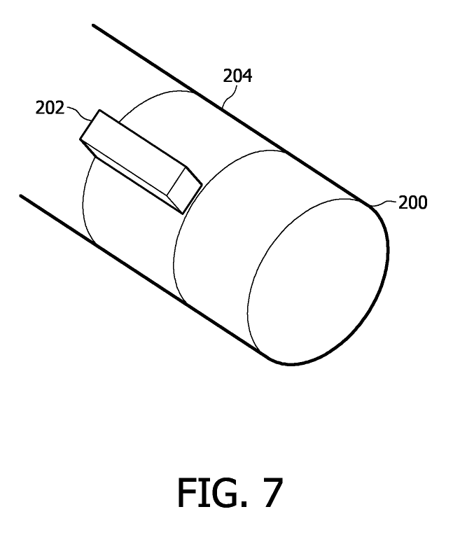

FIG. 7 schematically shows an embodiment of the puncture system applied to a body part of a person or an animal. Here, the puncture system is designed as an apparatus 202 for detecting a blood vessel underneath the surface of the skin of the body part 200 and for autonomously inserting a cannula into the blood vessel in response to detect at least the blood vessel's location. The apparatus 202 is further rigidly attached to a cuff 204 that provides fixing of the apparatus 202 to the body part 200. Typically, the cuff 204 or collar features a clearance which is at least partially covered by the apparatus 202 in order to provide direct access to the skin of the body part 200. The illustrated apparatus 202 at least comprises the cannula and corresponding cannula insertion means as well as actuation means for positioning and inserting the cannula into the blood vessel and further has an acquisition module for acquiring of blood vessel related data.

In typical implementations the needle or cannula can be connected to various reservoirs for either blood withdrawal or drug delivery. Hence, the needle or cannula may serve as injection needle or cannula or as withdrawal cannula. The embodiment illustrated in FIG. 6 provides a high degree of failure safety, since for e.g. blood withdrawal, the puncture system only has to be rigidly attached to the accessible body part 200 by means of the cuff 204. A cannula or needle insertion is then autonomously performed by the puncture system, thus allowing unskilled and untrained personnel to perform such invasive procedures.

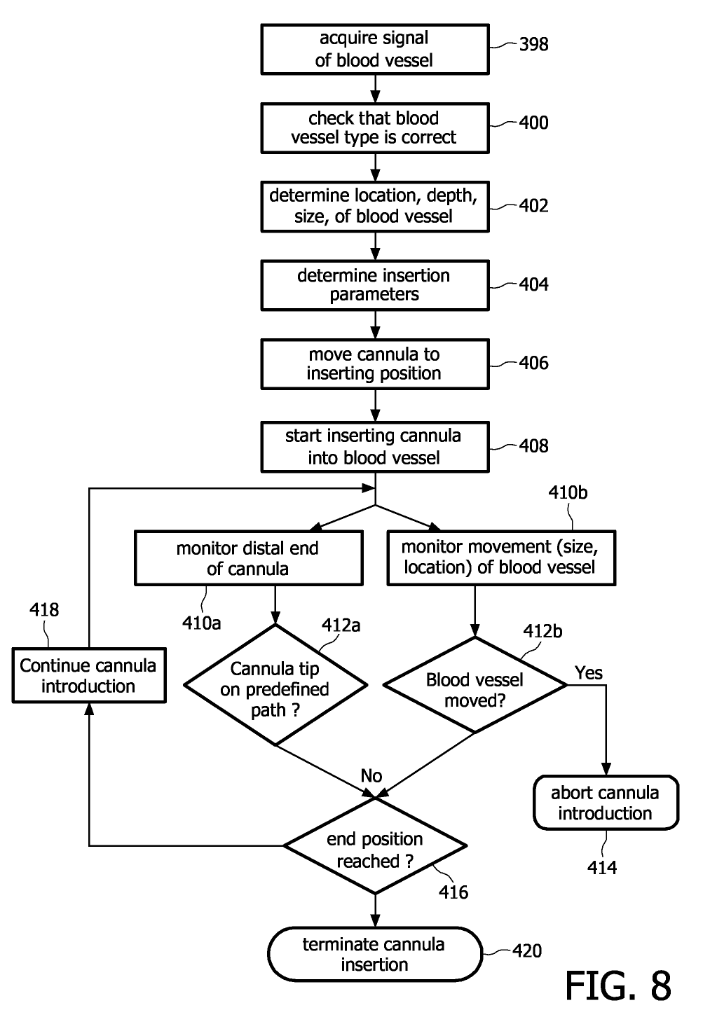

FIG. 8 is illustrative of a flowchart of determining a puncture and inserting position and for performing the needle or cannula insertion. In a first step 398 a signal of the blood vessel 102 is acquired by means of the acquisition module 108. The acquired data is then processed by means of the processing unit of the control unit 112 in order to check that the blood vessel type (artery or vein) is correct for the application. As a next step 400 the acquired data is further processed by means of the processing unit of the control unit 112 in order to determine e.g. location, depth, size, course and general geometric data of the blood vessel. Based on these blood vessel parameters representing blood vessel location and general properties of the blood vessel, in step 404 insertion parameters for the needle or cannula insertion are determined, preferably by means of an optimization procedure performed by the control unit 112. The insertion parameters represent at least the puncture location 124 of the blood vessel 102. Preferably, the insertion parameters also specify the inserting position 126 as well as the inserting direction given by the angle 119.

After the insertion parameters have been determined, the cannula is moved in step 406 to the insertion position. Hence, the needle or cannula is aligned along the inserting direction as well as laterally displaced to the inserting position 126. If the distal end 122 of the needle or cannula has reached the determined inserting position, in step 408 the insertion of the needle or cannula into the blood vessel starts. During this movement the following steps 410 through 420 are performed.

To follow needle insertion at least two alternative possibilities can be chosen:

A first possibility is as follows: During the advancing of the needle or cannula along the inserting direction 120, the distal end of the needle or cannula is monitored in step 410a by means of the acquisition module 108 and corresponding signal processing means. In the successive step 412a, the distal end of the needle or cannula or the needle or cannula tip's position is compared with the trajectory of the insertion path 128. In cases where the position of the distal end of the cannula 117 clearly deviates from the calculated insertion path 128, the method continues with step 414, where the needle or cannula introduction or insertion is aborted and the needle or cannula is withdrawn from the tissue in the opposite direction. Otherwise, if in step 412 the distal end of the cannula coincides with the insertion path 128 at least within a predefined margin, the method continues with step 416 where the position of the distal end of the needle or cannula is compared with the calculated end position inside the blood vessel 102.

A second possibility is as follows: During the advancing of the needle or cannula along the inserting direction 120, the location and size of the blood vessel 102 is monitored in step 410b by means of the acquisition module 108 and corresponding signal processing means. In the successive step 412b, the blood vessel location and size is compared with its original location and size just before starting the insertion. In cases where the position and size of the blood vessel clearly deviates from its original location and size, the method continues with step 414, where the needle or cannula introduction or insertion is aborted and the needle or cannula is withdrawn from the tissue in the opposite direction. Otherwise, if in step 412 the location and size of the blood vessel coincides with its original parameters at least within a predefined margin, the method continues with step 416 where the location and size of the blood vessel is compared with the original parameters of the blood vessel 102.

If the end position has not yet been reached, the method continues with step 418, where the needle or cannula introduction continues. Thereafter, the method returns to step 410a or 410b, where the position of the distal end of the needle or cannula 122 or the location and size of the blood vessel 102 is repeatedly monitored. It is to be noted that the movement of the needle or cannula is performed as a continuous movement, i.e. it is not performed stepwise as the single steps of the flowchart in FIG. 8 may suggest. The loop described by steps 410 through steps 418 continues as long as the end position of the cannula has not been reached. As soon as in step 416 the end position of the cannula has been detected, the procedure continues with step 420, where the cannula insertion is terminated.

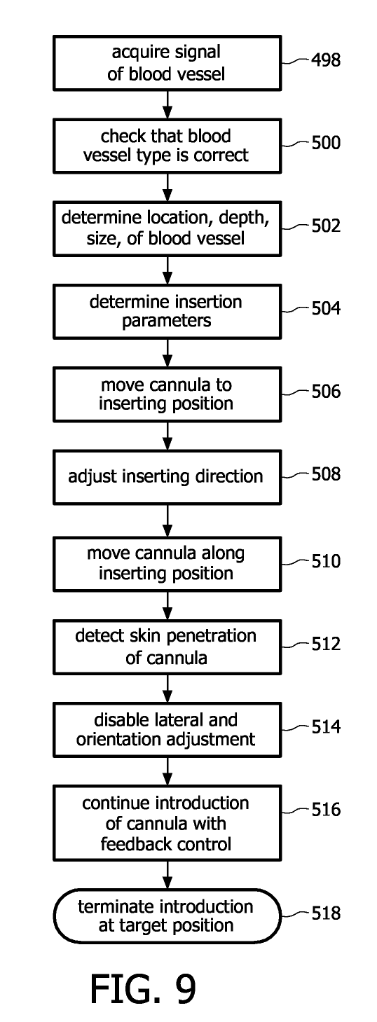

FIG. 9 is an illustrative flowchart and shows how the needle or cannula is introduced into the blood vessel 102 making use of a skin penetration detection of the needle or cannula. Here, steps 498 through 504 correspond to steps 400 through 404 of FIG. 8. In step 506 the needle or cannula is moved to the inserting position by a translational movement controlled by the needle or cannula control unit 114. Thereafter, in step 508 the inserting direction of the cannula 117 is adjusted according to the determined inserting angle 119. Alternatively, needle or cannula insertion may also be performed by making use of a predefined insertion angle, thus allowing to reduce the complexity of the puncture system. In step 510 the movement of the cannula along the inserting direction is started, thereby making use of position determination of the cannula's distal end or possible blood vessel movement for controlling the inserting motion.

In step 512 the system detects the penetration of the skin of the person by the cannula and in the subsequent step 514 all lateral and orientational movement or adjustment of the cannula are disabled. Detection of skin penetration can be performed by means of the acquisition module 108 and its corresponding signal processing means. Also, the skin penetration might be detected by means of a pressure sensor being implemented in the cannula mount 116. Disabling of lateral cannula movement serves as an effective means for injury prevention. After lateral adjustment of the cannula has been disabled in step 514, in step 516 introduction or insertion of the cannula into the blood vessel is continued by making use of the feedback control mechanism illustrated by the flowchart of FIG. 4. As soon as the estimated end position of the cannula has been reached the procedure stops in step 518.

In general, cannula or needle insertion described by the flowchart of FIG. 8 can be performed with feedback mechanism providing the actual location of the cannula's distal end or checking possible displacement of the blood vessel, preferably during insertion of the cannula. However, the entire needle or cannula insertion process may also be performed without such a feedback mechanism, allowing to reduce the complexity of the puncture system. In this case, after determination of the puncture location as well as determination of required blood vessel parameters, needle or cannula insertion can be autonomously or manually performed without a continuous needle or cannula or blood vessel tracking just by making use of the determined blood vessel parameters.

### LIST OF REFERENCE NUMERALS

100 puncture system
102 blood vessel
104 skin
106 tissue
108 acquisition module
110 detection system
112 control unit
114 cannula control
116 cannula mount
117 cannula
118 direction
119 angle
120 inserting direction
122 distal end of cannula
124 puncture location
126 inserting position
128 insertion path
200 body part
202 apparatus
204 cuff
300 vein
301 artery
302 imaging probe
303 skin surface

1-10. (canceled)

11. A puncture system (100) for inserting a cannula (117) into a blood vessel (102) of a person or animal comprising:
location determination means for determining at least one location of the blood vessel,
processing means for determining a puncture location (124) of the blood vessel depending on the output of the location determination means,
cannula insertion means for guiding the insertion of the cannula into the identified blood vessel,
actuation means adapted to autonomously move the cannula insertion means into an insertion position corresponding to the puncture location.

12. A puncture system according to claim 11,
characterized in that it further comprises blood vessel identification means for distinguishing arteries from veins and the blood vessel identification means is adapted to measure the direction of the blood flow.

13. A puncture system according to claim 11,
characterized in that it further comprises blood vessel identification means for distinguishing arteries from veins and the blood vessel identification means is adapted to analyse the flow characteristics of the blood.

14. A puncture system according to claim 11,
characterized in that it further comprises blood vessel identification means for distinguishing arteries from veins and the blood vessel identification means is adapted to carry out an identification on the basis of mechanical palpation.

15. A puncture system according to claim 11,
characterized in that it further comprises blood vessel identification means for distinguishing arteries from veins and the blood vessel identification means is adapted to measure the oxygen content in the blood.

16. A puncture system according to claim 11,
characterized in that,
the insertion means is adapted to
a) fix the cannula, said insertion means being moveable along an inserting direction (120) and at least a second direction (118) being substantially non-parallel to the inserting direction, and
b) to insert the distal end (122) of the cannula into the blood vessel at the puncture location.

17. A puncture system according to claim 11,
characterized in that
the location determination means are adapted to track the location of the cannula's distal end (122) during insertion of the cannula (117) and further comprise control means for controlling the movement of the cannula in response to the tracking of the cannula's distal end.

18. A puncture system according to claim 11,
characterized in that
the location determination means is adapted to track the location and size of the blood vessel during insertion of the cannula (117) and further comprise control means for controlling the movement of the cannula in response to the tracking of the blood vessel parameters.

19. A puncture system according to claim 11,
characterized in that the actuation means further controls the insertion of the distal end (122) of the cannula into the blood vessel at the puncture location.

20. A computer program product for a puncture system (100) for inserting a cannula into a blood vessel (102) of a person or animal, the computer program product comprising instructions being executable by processing means of the puncture system for performing the steps of:
a) determining at least one blood vessel parameter of the blood vessel by processing the output of location determination means, the at least one blood vessel parameter being at least representative of the blood vessel's location,
b) identifying whether the blood vessel is an artery or a vein,
c) determining a puncture location (124) of the blood vessel by processing of the at least one blood vessel parameter,
d) calculating a driving signal for driving actuation means (114) to insert the cannula's distal end (122) into the blood vessel at the puncture location.
